(19) United States
(12) Patent Application Publication
(10) Pub. No.: US 2024/0076706 A1
(43) Pub. Date: Mar. 7, 2024

(54) 1,3/1,4-XYLANASE MLX1034, ITS GENE AND APPLICATIONS THEREOF

(71) Applicant: SHAN DONG UNIVERSITY, Jinan (CN)

(72) Inventors: Yuzhong ZHANG, Jinan (CN); Fang ZHAO, Jinan (CN); Xiulan CHEN, Jinan (CN); Haining SUN, Jinan (CN); Xiaoyan SONG, Jinan (CN); Pingyi LI, Jinan (CN)

(73) Assignee: SHAN DONG UNIVERSITY, Jinan (CN)

(21) Appl. No.: 18/261,847

(22) PCT Filed: Dec. 7, 2021
(86) PCT No.: PCT/CN2021/136034

§ 371 Date: Jul. 18, 2023

(30) Foreign Application Priority Data
Jan. 20, 2021 (CN) 202110074436.5

(51) Int. Cl. C12P 19/14; C12N 9/24

(52) U.S. Cl. CPC C12P 19/14; C12N 9/248; C12R 2001/20

(57) ABSTRACT

MLX1034 is from Polaribacter sp. Q13, and has the amino acid sequence of the 1,3/1,4-xylanase MLX1034 is listed in SEQ ID NO.1; a nucleotide sequence of the gene is listed in SEQ ID NO.2; the 1,3/1,4-xylanase MLX1034 in the invention is capable of efficiently and specifically degrading 1,3/1,4-xylan and producing xylooligosaccharides with DP values above one; in addition, the physical and chemical properties of the 1,3/1,4-xylanase MLX1034 are stable enough to hydrolyze 1,3/1,4-xylan at room temperature; the 1,3/1,4-xylanase MLX1034 is suitable for the industrial production of red algal xylooligosaccharides at low energy costs.

### FIELD

The invention relates to the 1,3/1,4-xylanase MLX1034, gene and applications thereof, belonging to the technical field of biotechnology.

### BACKGROUND

Xylan, the main component of hemicellulose, is the second most abundant biopolymer after cellulose. Xylans in the cell walls of land plants are complex heteropolymers, known as heteroxylans. The backbones of heteroxylans are composed of β-1,4-linked xylose and are generally decorated with α-L-arabinofuranose, 4-O-methyl-glucuronic acid, acetate, or ferulic acid. Thus, a large diversity of xylanolytic enzymes is required for the complete hydrolysis of heteroxylans, among which endolytic 1,4-xylanases play a leading role.

1,4-xylooligosaccharides are composed of two to seven β-1,4-linked xylose residues. Extensive studies have suggested that 1,4-xylooligosaccharides are capable of preferentially stimulating the growth of prebiotic bacteria (e.g., Bifidobacterium) in the gastrointestinal tract, modulating gut microbiota, enhancing immune function and exhibiting beneficial effects on reducing hypertension, hyperglycemia and cholesterol, and anti-cancer. Therefore, 1,4-xylooligosaccharides are widely used in industries of food, medicine, cosmetics and feed additives. At present, 1,4-xylooligosaccharides are mainly prepared from corn cobs, wheat brans and bagasse by enzymatic or chemical catalysis.

Xylans in the ocean are mainly from the cell walls of red and green algae with structures different from heteroxylans in land plants. Xylans from marine algae include 1,3-xylan, 1,4-xylan and mixed-linkage 1,3/1,4-xylan, all of which are linear homoxylans composed of only xylose residues. 1,3/1,4-xylan is the main polysaccharide component of many red algae in the orders Palmariales and Nemaliales. Amongst, Palmaria palmata (also called dulse) from the order Palmariales is a famous economic seaweed, which is widely distributed on the northern coasts of the Atlantic and Pacific oceans. Due to the abundance of dietary fiber, protein, vitamins, and antioxidant and anti-inflammatory materials, Palmaria palmata becomes a popular red sea vegetable, especially in Europe and Japan. Water-soluble 1,3/1,4-xylan is known from Palmaria palmata, which contains both β1,3- and β1,4-linkages at a ratio of approximately 1:4 with an irregular distribution of single β1,3-linkages along the β1,4-linked backbone. 1,3/1,4-xylan from Palmaria palmata has been commercialized.

Due to the huge primary productivity of marine red algae, there are huge 1,3/1,4-xylan resources in the ocean. With the 1,3/1,4-xylan as raw materials, novel branchless xylooligosaccharides containing β-1,3-linkages can be prepared, which are different from the known 1,4-xylooligosaccharides. The novel xylooligosaccharides have a good application prospect. However, there is still a lack of efficient 1,3/1,4-xylanases to prepare xylooligosaccharides from 1,3/1,4-xylan.

### SUMMARY

In view of the state of the art, the present invention provides the 1,3/1,4-xylanase MLX1034, gene and applications thereof.

The technical solution of the present invention is as follows:

The present invention provides a 1,3/1,4-xylanase, MLX1034. The amino acid sequence of the 1,3/1,4-xylanase is listed in SEQ ID NO.1.

According to the present invention, preferably, the 1,3/1,4-xylanase MLX1034 is an endolytic enzyme from Polaribacter sp. Q13. Polaribacter sp. Q13 is deposited in China Center for Type Culture Collection at Jialuoshan, Bayi Road, Wuhan City, Hubei Province on Dec. 28, 2020. The deposit number is CCTCC M 2020985.

When the 1,3/1,4-xylanase MLX1034 degrades 1,3/1,4-xylan, the end products are xylooligosaccharides with degrees of polymerization (DP) above one and the main product is xylohexaose.

The nucleotide sequence of the gene encoding the above 1,3/1,4-xylanase MLX1034 is listed in SEQ ID NO.2.

According to the present invention, preferably, the gene encoding the 1,3/1,4-xylanase MLX1034 is cloned from the genomic DNA of Polaribacter sp. Q13. Polaribacter sp. Q13 is deposited in China Center for Type Culture Collection at Jialuoshan, Bayi Road, Wuhan City, Hubei Province on Dec. 28, 2020. The deposit number is CCTCC M 2020985.

A recombinant expression vector incorporating the gene encoding the 1,3/1,4-xylanase MLX1034.

According to the present invention, preferably, the recombinant expression vector is constructed with the plasmid pET-22b.

According to the present invention, preferably, the recombinant expression vector is transformed into a host cell to produce recombinant cells.

According to the present invention, preferably, the host cell is Escherichia coli.

According to the present invention, preferably, the E. coli is E. coli BL21(DE3).

A recombinant cell incorporating the gene encoding the 1,3/1,4-xylanase MLX1034.

A recombinant expression strain, the strain is obtained by cultivating the recombinant cell.

According to the present invention, preferably, the recombinant cell is cultivated in the Luria-Bertani (LB) medium at 37° C. in shake flasks to an optical density at 600 nm (OD_{600 nm}) of approximately 0.8˜1.0.

According to the present invention, preferably, after OD_{600 nm} reaches 0.8˜1.0, IPTG is added to induce protein expression and the recombinant expression strain is collected.

According to the present invention, preferably, the 1,3/1,4-xylanase MLX1034 is derived from the recombinant expression strain.

According to the present invention, applications of the 1,3/1,4-xylanase MLX1034 in the degradation of 1,3/1,4-xylan.

A method for the degradation of 1,3/1,4-xylan. In detail, the gene, whose nucleotide sequence is listed in SEQ ID NO.2, is inserted into the plasmid pET-22b to obtain the recombinant expression vector. The recombinant expression vector is transformed into a host cell to obtain recombinant cells. The recombinant cells are cultivated in fermentation medium and produce the 1,3/1,4-xylanase MLX1034. 1,3/1,4-xylan is hydrolyzed by the 1,3/1,4-xylanase MLX1034.

A method for the preparation of xylooligosaccharides. In detail, the gene, whose nucleotide sequence is listed in SEQ ID NO.2, is inserted into a plasmid to obtain the recombinant expression vector. The recombinant expression vector is transformed into a host cell to obtain recombinant cells. The recombinant cells are cultivated in fermentation medium and produce the 1,3/1,4-xylanase MLX1034. 1,3/1,4-xylan is hydrolyzed by the 1,3/1,4-xylanase MLX1034 to obtain xylooligosaccharides.

According to the present invention, preferably, the DP values of the xylooligosaccharides are above one.

According to the present invention, preferably, the main component of the xylooligosaccharides is xylohexaose.

According to the present invention, preferably, the plasmid is pET-22b.

According to the present invention, preferably, the host cell is E. coli, further preferably E. coli BL21(DE3).

According to the present invention, preferably, the recombinant cell is cultivated in LB medium at 37° C. in shake flasks to an OD_{600 nm} of approximately 0.8˜1.0. Preferably, after OD_{600 nm} reaches 0.8˜1.0, IPTG is added to induce protein expression and the cells are collected by centrifugation.

The technical characteristics of the present invention is as follows:

Polaribacter sp. Q13 according to the present invention is isolated from the surface of red algae collected from Nelson Island, Antarctic. Based on the genomic and secretomic data, MLX1034 is identified as an extracellular 1,3/1,4-xylanase secreted by Polaribacter sp. Q13. The gene MLX1034 is 1017 bp in length and encodes 338 amino acid residues. Sequence analysis indicates that MLX1034 contains an N-terminal signal peptide (Met1-Ser27) with a calculated molecular weight of 39.1 kDa. MLX1034 have a single catalytic domain of glycoside hydrolase (GH) family 26, which is a previously unreported 1,3/1,4-xylanase with a new amino acid sequence. To obtain the recombinant strain, the gene MLX1034 is cloned from the genomic DNA of Polaribacter sp. Q13 and incorporated into E. coli. The 1,3/1,4-xylanase MLX1034 is expressed in the recombinant strain. The 1,3/1,4-xylanase MLX1034 is inactive on 1,4-xylan, displays little activity on 1,3-xylan, but efficiently hydrolyzes 1,3/1,4-xylan, indicating that it is a 1,3/1,4-xylan-specific xylanase. The 1,3/1,4-xylanase MLX1034 shows the maximum activity at 40° C. and pH 7.0 in the phosphate buffered saline (PBS), indicating that it is a neutral and mesophilic enzyme. High performance liquid chromatography (HPLC) of the hydrolysis products released from 1,3/1,4-xylan by MLX1034 indicates that MLX1034 is a strict endolytic xylanase. The main product of MLX1034 at each time point is xylohexaose with no xylose, indicating that the 1,3/1,4-xylanase MLX1034 is an effective tool enzyme to prepare xylooligosaccharides from 1,3/1,4-xylan.

### ADVANTAGEOUS EFFECTS OF THE INVENTION

The 1,3/1,4-xylanase MLX1034 according to the present invention is from Polaribacter sp. Q13. The 1,3/1,4-xylanase MLX1034 can be easily produced in E. coli and purified to high purity. The 1,3/1,4-xylanase MLX1034 can efficiently and specifically degrade 1,3/1,4-xylan and generate xylooligosaccharides with DP values of two to eight. In addition, the 1,3/1,4-xylanase MLX1034 is stable at room temperature and suitably used in red algal xylooligosaccharides preparation at a low cost.

In addition, when the 1,3/1,4-xylanase MLX1034 degrades 1,3/1,4-xylan, the main product at each time point is xylohexaose with no xylose. Thus, the 1,3/1,4-xylanase MLX1034 has significant advantages in technics optimization.

### BRIEF DESCRIPTION OF THE DRAWINGS

FIG. 1 SDS-PAGE analysis of the recombinant 1,3/1,4-xylanase MLX1034. M, protein marker.

FIG. 2 Effect of temperature on the activity of the 1,3/1,4-xylanase MLX1034.

FIG. 3 Effect of temperature on the stability of the 1,3/1,4-xylanase MLX1034.

FIG. 4 Effect of pH on the activity of the 1,3/1,4-xylanase MLX1034.

FIG. 5 Effect of pH on the stability of the 1,3/1,4-xylanase MLX1034.

FIG. 6 High-performance liquid chromatography (HPLC) analysis of the released xylooligosaccharides from 1,3/1,4-xylan by the 1,3/1,4-xylanase MLX1034. The reaction mixture without the 1,3/1,4-xylanase MLX1034 was used as the control.

### DETAILED DESCRIPTION OF THE PREFERRED EMBODIMENTS

The present invention will be further described below through drawings and embodiments. It is to be understood that the invention is not limited to the embodiments described below, and any form of modifications and/or changes made to the invention are intended to fall within the scope of the invention.

The materials used in the present invention are described below.

A bacterial strain, Polaribacter sp. Q13, has been deposited in China Center for Type Culture Collection at Jialuoshan, Bayi Road, Wuhan City, Hubei Province on Dec. 28, 2020. The deposit number is CCTCC M 2020985.

1,3/1,4-xylan from Palmaria palmata was purchased from Elicityl (France). The proportion between β-1,3- and β-1,4-linkages is approximately 1:4.

1,3-xylan was extracted from Caulerpa lentillifera.

Wheat arabinoxylan and beech wood xylan were purchased from Megazme (Ireland).

TYS broth medium contained 0.5% (w/w) tryptone and 0.1% (w/w) yeast extract in artificial seawater [3.0% (w/w) sea salts (Sigma, USA) in ddH₂O]. The pH was adjusted to 7.8.

TYS agar medium contained 0.5% (w/w) tryptone, 0.1% (w/w) yeast extract and 1.5% (w/w) agar in artificial seawater. The pH was adjusted to 7.8.

Luria-Bertani (LB) medium contained 1.0% (w/w) tryptone, 0.5% (w/w) yeast extract and 1.0% (w/w) NaCl in ddH₂O.

LB agar medium contained 1.0% (w/w) tryptone, 0.5% (w/w) yeast extract, 1.0% (w/w) NaCl and 1.5% (w/w) agar in ddH₂O.

1,3/1,4-xylan agar medium contained 0.2% (w/w) 1,3/1,4-xylan and 1.5% (w/w) agar in artificial seawater. The pH was adjusted to 7.8.

### EXAMPLE 1 ISOLATION OF POLARIBACTER SP. Q13

Red algal samples were collected from Nelson Island, Antarctica. These algal samples were washed with sterile artificial seawater to obtain epiphytic bacteria from the algal surface. After gradient dilution (10⁻²-10⁻⁶), the bacteria-containing seawater was spread on TYS agar plates. Then the bacteria were incubated at 20° C. until detectable colonies formed. Morphologically different colonies were selected, purified with TYS agar, and then cryopreserved at −80° C. in TYS broth supplemented with glycerol [TYS broth: glycerol=3:7 (v/v)]. Furthermore, the purified strains were cultivated on 1,3/1,4-xylan agar plates to screen 1,3/1,4-xylan-utilizing bacteria. Finally, one bacterial strain, designated as Q13, was found to grow on the 1,3/1,4-xylan agar plate.

After 3˜4 days cultivation on the 1,3/1,4-xylan agar plate, colonies of strain Q13 were yellow, moist and circular with regular edges and smooth surfaces. Cells of strain Q13 were Gram-stain-negative. Strain Q13 was identified as a species of the genus Polaribacter (Polaribacter sp.).

Polaribacter sp. Q13 has been deposited in China Center for Type Culture Collection at Jialuoshan, Bayi Road, Wuhan City, Hubei Province on Dec. 28, 2020. The deposit number is CCTCC M 2020985.

### EXAMPLE 2 CLONING OF THE GENE MLX1034 AND CONSTRUCTION OF THE RECOMBINANT PLASMID pET-22b-MLX1034

1. Genomic DNA extraction and whole genome sequencing of Polaribacter sp. Q13. Polaribacter sp. Q13 was cultivated in TYS broth at 20° C. and 180 rpm for 24 h. Then the bacterial cells were collected and the genomic DNA was extracted using a bacterial genomic DNA isolation kit (BioTeke, China) according to the manual instruction. The whole-genome sequencing was carried out at the Beijing Genomics Institute (BGI; Beijing, China).
2. Primer design and synthesis. Based on the genomic data of Polaribacter sp. Q13, the nucleotide sequence of the gene MLX1034 and the amino acid sequence of its product (MLX1034) were obtained. Using SignalP-5.0 (http://www.cbs.dtu.dk/services/SignalP/), the signal peptide of MLX1034 was predicted as Met1-Ser27. After deleting the predicted signal peptide, a forward primer (F: 5′-AAGAAGGAGATATACATATGCAAGAGGTAAAACCTCGTTTT-3′) and a reverse primer (R: 5′-TGGTGGTGGTGGTGCTCGAGTTTATTATTAAGGTGAATAAA-3′) were designed and synthesized at BGI (Beijing, China).
3. PCR amplification and PCR product recovery. PCR amplification was performed using primers F and R with the genomic DNA of Polaribacter sp. Q13 as the template. The PCR program was determined as pre-denaturation at 95° C. for 2 min, followed by 30 cycles of PCR (denaturation at 95° C. for 30 s, annealing at 55° C. for 20 s and extension at 72° C. for 45 s), and a final extension step at 72° C. for 10 min. The 50 μL PCR mixture contained 29 μL sterile ddH₂O, 10 μL Trans Star Fast Pfu buffer (TransGen, China), 5 μL dNTP, 2 μL primer F, 2 μL primer R, 1 μL genomic DNA of strain Q13 and 1 μL Trans Star Fast Pfu DNA polymerase (TransGen, China). The amplification products were purified by the 1.0% (w/w) agarose gel electrophoresis and a gel extraction kit (Omega, USA), and the MLX1034 fragment was obtained.
4. Construction of the recombinant plasmid pET-22b-MLX1034. The pET-22b vector (Novagen, USA) was digested with the restriction enzymes NdeI and XhoI and the linear product was purified by the 1.0% (w/w) agarose gel electrophoresis and a gel extraction kit (Omega, USA). Then, the obtained MLX1034 fragment was inserted into the digested pET-22b with the In-Fusion HD Cloning Kit (TaKaRa, Japan). The 2.5 μL reaction mixture contained 1 μL MLX1034 fragment, 1 μL digested pET-22b and 0.5 μL In-Fusion.

Using the heat shock method described in “Molecular cloning: A laboratory manual”, the assembled vector was transformed into the competent Escherichia coli DH5α cells (TransGen, China). In detail, 2.5 μL ligation products were added into 50 μL competent E. coli DH5α cells. After an incubation in ice for 30 min, the mixture is placed at 42° C. for 90 s and then placed back in ice for 10 min. 200 μL LB was added and the and the transformed cells were incubated at 37° C. for 1 h. The resultant cells were spread on the LB agar plate containing 100 μg/mL ampicillin and incubated at 37° C. overnight. Single colonies were picked and cultivated in LB containing 100 μg/mL ampicillin at 37° C. overnight. The cells were collected and sent to BGI (Beijing, China) for plasmid extraction and gene sequencing.

The result of gene sequencing showed that MLX1034 was successfully inserted into the NdeI-XhoI restriction site of pET22b with no substitution, deletion or insertion mutations. The recombinant plasmid was named pET-22b-MLX1034.

### EXAMPLE 3 EXPRESSION AND PURIFICATION OF THE 1,3/1,4-XYLANASE MLX1034

1. Expression of the 1,3/1,4-xylanase MLX1034 in E. coli BL21(DE3). The recombinant plasmid pET-22b-MLX1034 was transformed into the competent E. coli BL21(DE3) cells (TransGen, China) with the heat shock method as mentioned above. The resultant cells were spread on the LB agar plate containing 100 μg/mL ampicillin and cultivated at 37° C. overnight. A single colony was picked and cultivated in LB containing 100 μg/mL ampicillin at 37° C. overnight, obtaining seed culture. The seed culture (1%, v/v) was inoculated into LB containing 100 μg/mL ampicillin and cultivated at 180 rpm and 37° C. to an optical density (OD) at 600 nm of approximately 0.8˜1.0. Then, 0.1 mM isopropyl-D-thiogalactopyranoside (IPTG) was added to the culture as an inducer, and the culture was incubated at 18° C. and 120 rpm for 16 h. The induced cells were harvested by centrifugation (7,000 rpm for 5 min at 4° C.).
2. Purification of the 1,3/1,4-xylanase MLX1034. The obtained cells were resuspended in the lysis buffer (50 mM Tris-HCl, 100 mM NaCl, pH 8.0) and lysed by a pressure crusher. After centrifugation (12,000 rpm for 60 min at 4° C.), the supernatant, namely crude enzyme solution, was collected.

The crude enzyme solution was loaded onto the nickel affinity column (GE Healthcare, USA), which was pre-equilibrated with the lysis buffer. Then, the nickel affinity column was washed with 10 column volumes of the wash buffer (50 mM Tris-HCl, 100 mM NaCl, 10 mM imidazole, pH 8.0). The target protein (MLX1034) was eluted with the elution buffer (50 mM Tris-HCl, 100 mM NaCl, 250 mM imidazole, pH 8.0).

The obtained sample was concentrated and loaded onto the gel filtration (GF) column (Superdex 200 Prep grade; GE Healthcare, USA), which was pre-equilibrated with the GF buffer (10 mM Tris-HCl, 100 mM NaCl, pH 8.0). The sample was then eluted with the GF buffer. Samples of the target protein peak were collected and stored at −80° C. with the addition of 10% (v/v) glycerol for further use.

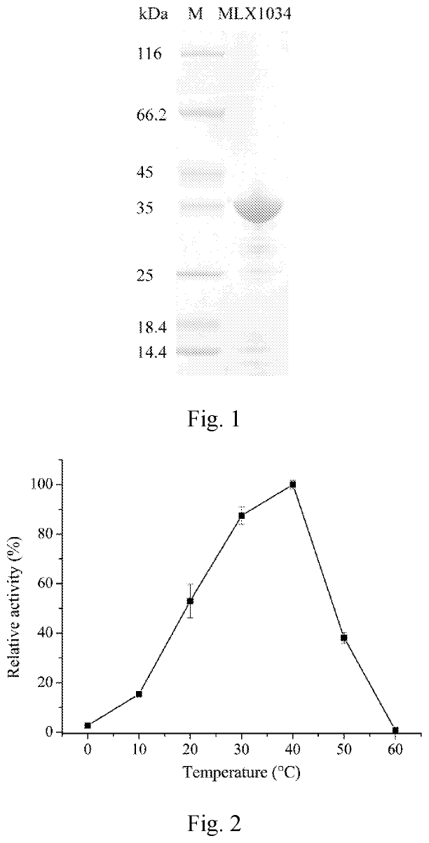

The purity and molecular weight of the purified MLX1034 were determined with SDS-PAGE. The result is shown in FIG. 1.

As shown in FIG. 1, the purified MLX1034 exhibited an apparent molecular weight of approximately 35.0 kDa, consistent with its theoretical molecular weight (39.1 kDa). Therefore, MLX1034 was successfully expressed in E. coli BL21 (DE3).

### EXAMPLE 4 ENZYMATIC PROPERTIES OF THE 1,3/1,4-XYLANASE MLX1034

The standard reaction mixture contained 900 μL xylan [10 mg/mL in 20 mM phosphate-buffered saline (PBS)] and 100 μL enzyme solution of the 1,3/1,4-xylanase MLX1034.

The xylanase activity was determined by the dinitrosalicylic acid (DNS) method. The reaction mixture was incubated at 40° C. for 10 min. After incubation, the reaction was terminated by the addition of 100 μL DNS. Then the reaction mixture was boiled at 100° C. for 5 min for coloring. After the addition of 500 μL ddH₂O, the OD_{550 nm} was measured. The reaction mixture supplemented with the inactivated MLX1034 was used as the control. One unit of enzyme activity (1 U) is defined as the amount of enzyme required to release 1 μmol xylose per min. A standard curve was generated with different concentrations of xylose.

Enzymatic properties of the 1,3/1,4-xylanase MLX1034 were analyzed according to the method.
1. Substrate specificity analysis. The substrate specificity of the 1,3/1,4-xylanase MLX1034 was analyzed with 1,3/1,4-xylan, 1,3-xylan and 1,4-xylans (including wheat arabinoxylan and beech wood xylan) as the substrates. The result is shown in Table 1.

As shown in Table 1, the 1,3/1,4-xylanase MLX1034 efficiently hydrolyzed 1,3/1,4-xylan (224.0 U/mg) with extremely low activity on 1,3-xylan (0.04 U/mg) and no activity on 1,4-xylans (including wheat arabinoxylan and beech wood xylan).

TABLE 1
The substrate specificity of the 1,3/1,4-xylanase MLX1034.

| Substrate | Specific activity (U/mg) | |
|---|---|---|
| 1,3/1,4-xylan | 224.0 ± 13.5 | (100%) |
| 1,3-xylan | 0.04 ± 0.00 | (0.02%) |

| | |
|---|---|
| Wheat arabinoxylan | Not detectable |
| Beech wood xylan | Not detectable |

2. Effect of temperature on enzymatic activity and stability. To determine the effect of temperature on the activity of the 1,3/1,4-xylanase MLX1034, 1,3/1,4-xylan was used as the substrate. Reaction mixtures were separately incubated at 0° C., 10° C., 20° C., 30° C., 40° C., 50° C. and 60° C. for 10 min, and the activities of the 1,3/1,4-xylanase MLX1034 at different temperatures were determined. The maximum activity was taken as 100% to calculate the relative activities. The result is shown in FIG. 2.

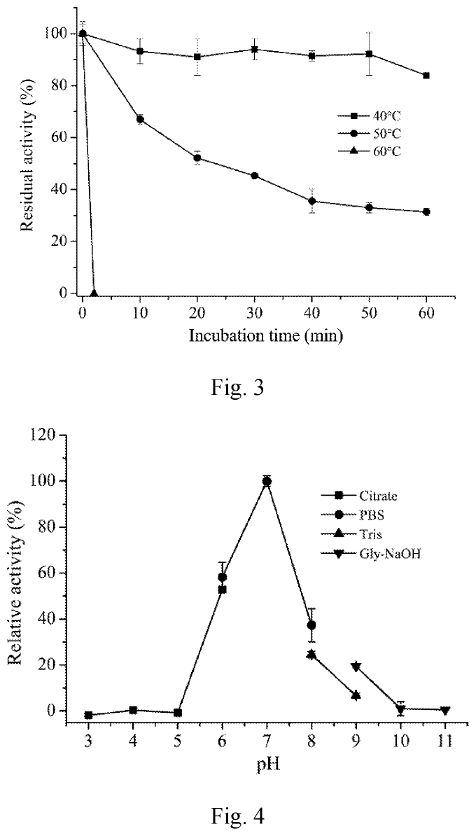

To determine the effect of temperature on the stability of the 1,3/1,4-xylanase MLX1034, it was separately incubated at 40° C., 50° C. and 60° C. for 60 min. During incubation, the residual activities were determined every 10 min with 1,3/1,4-xylan as the substrate. The specific activity of the 1,3/1,4-xylanase MLX1034 without incubation (0 min) was taken as 100% to calculate the residual activities. The result is shown in FIG. 3.

As shown in FIGS. 2 and 3, the 1,3/1,4-xylanase MLX1034 exhibited more than 80% activity at 30˜45° C. with the maximum activity at 40° C. It was relatively stable at 40° C., retaining more than 80% residual activities during 60 min incubation. After 60 min incubation at 50° C., the residual activity of the 1,3/1,4-xylanase MLX1034 decreased to less than 40%. It was extremely unstable at 60° C. and rapidly deactivated within 5 min.
3. Effect of pH on enzymatic activity and stability. To determine the effect of pH on the activity of the 1,3/1,4-xylanase MLX1034, reactions were carried out at 40° C. for 10 min under different pH values from 3.0 to 11.0 with 1,3/1,4-xylan as the substrate. The buffers used were 20 mM citrate buffer at pH 3.0˜6.0, 20 mM PBS at pH 6.0˜8.0, 20 mM Tris-HCl at pH 8.0˜9.0, and 20 mM glycine-NaOH at pH 9.0˜11.0. The maximum activity was taken as 100% to calculate the relative activities. The result is shown in FIG. 4.

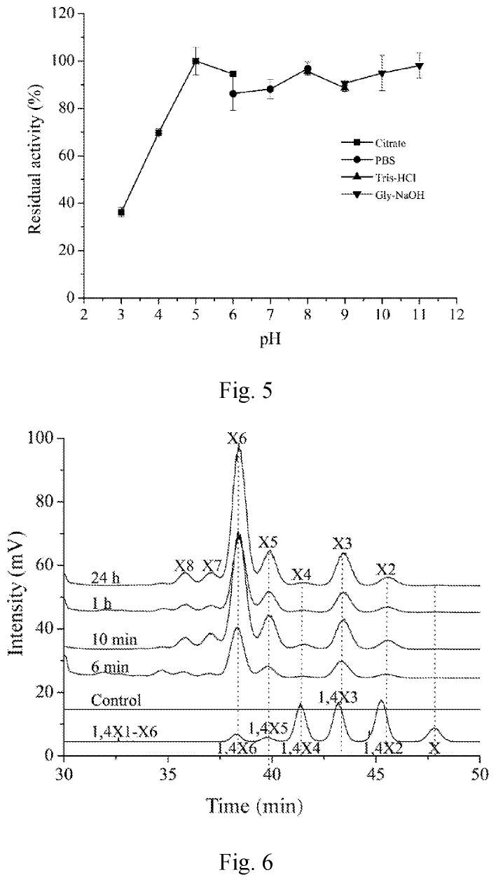

To determine the effect of temperature on the stability of MLX1034, each of the buffers with different pH values was mixed with the 1,3/1,4-xylanase MLX1034 at a volume ratio of 49:1. The buffers used were 20 mM citrate buffer at pH 3.0˜6.0, 20 mM PBS at pH 6.0˜8.0, 20 mM Tris-HCl at pH 8.0˜9.0, and 20 mM glycine-NaOH at pH 9.0˜11.0. After incubation at 60° C. for 1 h, the residual activities of the 1,3/1,4-xylanase MLX1034 were determined with 1,3/1,4-xylan as the substrate. The maximum activity was taken as 100% to calculate the residual activities. The result is shown in FIG. 5.

As shown in FIGS. 4 and 5, the 1,3/1,4-xylanase MLX1034 exhibited the highest activity at pH 7.0. It was unstable under acidic conditions (pH 3.0˜4.0) but stable at pH 5.0˜11.0.

### EXAMPLE 5 MODE OF ACTION ANALYSIS OF THE 1,3/1,4-XYLANASE MLX1034

A reaction mixture containing 1,3/1,4-xylan (5 mg/mL in 20 mM PBS, pH 7.0) and the 1,3/1,4-xylanase MLX1034 at a volume ratio of 99:1 was incubated at 30° C. At the reaction time of 6 min, 10 min, 1 h and 24 h, samples were taken out separately and boiled for 5 min. Then samples were filtered with a 0.22 μM filter, obtaining the degradation products.

The degradation products were analyzed using HPLC equipped with an evaporative light scattering detector (ELSD) and a Superdex 30 Increase 10/300 GL column (GE Healthcare, USA). The mobile phase was ddH₂O. A mixture of xylose and 1,4-xylooligosaccharides (1,4X2-X6) was used as the marker. The result is shown in FIG. 6.

As shown in FIG. 6, the 1,3/1,4-xylanase MLX1034 is an endolytic xylanase. The degradation products were xylooligosaccharides with DP values above one with xylohexaose as the main product. At each time point of the reaction within 24 h, only xylooligosaccharides (DP>2) were produced with no xylose.

What is claimed is:

1. A 1,3/1,4-xylanase MLX1034 protein having the amino acid sequence shown as SEQ ID NO.1.

2. The 1,3/1,4-xylanase MLX1034 protein according to claim 1, wherein the protein is an endolytic xylanase isolated from Polaribacter sp. Q13. Polaribacter sp. Q13 is deposited in China Center for Type Culture Collection at Jialuoshan, Bayi Road, Wuhan City, Hubei Province on Dec. 28, 2020. The deposit number is CCTCC M 2020985.

3. The 1,3/1,4-xylanase MLX1034 protein according to claim 1, wherein the protein is encoded by a gene having the nucleotide sequence shown as SEQ ID NO.2.

4. The 1,3/1,4-xylanase MLX1034 protein according to claim 3, wherein the gene is cloned from the genomic DNA of Polaribacter sp. Q13. Polaribacter sp. Q13 is deposited in China Center for Type Culture Collection at Jialuoshan, Bayi Road, Wuhan City, Hubei Province on Dec. 28, 2020. The deposit number is CCTCC M 2020985.

5. An expression vector comprising the gene of claim 3 that encodes the 1,3/1,4-xylanase MLX1034 protein.

6. The expression vector according to claim 5, wherein a recombinant cell comprising the expression vector.

7. A method for degrading 1,3/1,4-xylan and preparing ylooligosaccharides by utilizing a 1,3/1,4-xylanase MLX1034 protein having the amino acid sequence shown as SEQ ID NO.1 that is encoded by a gene having the nucleotide sequence shown as SEQ ID NO.2.


SEQUENCE LISTING (2 sequences)

SEQ ID NO: 1 - protein, 338 aa, Polaribacter sp. Q13
MIKLIGSLFLGKLSLIILLLFLQNGFSQEVKPRFNYNAKYEPEKGIYHGAGQDKNGFHDYVNAVGQDKMPAIYMTYVNITSPVKRIERWGKSLKQVLDSLPKGIMPQIGLGFTGGKDTGAGLDKEVADGKYNKQLQAFYKVLLDLDRPSFTRIGYEFEGDWNGYSPESFKKVFITISKAFKEKNIKSATVWCSGGGSADFISLDKLMEYYPGDEYVDWWGIDIFSPEEFDHIGLQNFFDAAHIHKRPVMIGECTPRFVGVLDGETSWGKWFKPFFRMLHDTPGIKAFCYINWDWEYWSNRNGFPWHDWKDARIEKNPFVLEAYKKEMEKPLFIHLNNK

SEQ ID NO: 2 - DNA, 1017 bp, Polaribacter sp. Q13
atgataaaattaataggttcattatttttaggtaagttaagtttaattattctattgttgtttttgcaaaatggatttagccaagaggtaaaacctcgttttaattacaatgctaaatacgaaccagaaaaaggaatttatcacggtgctggtcaagataaaaatgggtttcacgattatgtaaatgctgtaggacaagataaaatgcctgcaatttatatgacgtatgttaatataacttcaccggtaaaaaggattgagcgttggggaaaaagtttaaaacaggttttagatagtttgcccaaagggataatgcctcaaattggtttagggtttactggcggtaaggatactggagcaggtttggataaagaagtcgctgatggaaaatataacaaacaattacaagcattttataaagtgcttttagatttagacagaccctcttttactagaattggttatgagtttgaaggcgattggaacggctattcacctgaaagttttaaaaaagtgttcatcactatttcaaaggcatttaaagaaaagaatataaaatcggctacggtttggtgctcaggtgggggctctgcagattttataagtcttgataagttgatggaatactatcctggtgatgaatatgtagattggtggggtatcgatatttttagtcctgaagaatttgatcatattggtttacaaaacttttttgatgcagcgcatattcataaaagacccgttatgattggcgagtgtactccacgatttgtaggtgttttagatggtgaaacttcttggggaaaatggttcaaacctttttttagaatgttacatgataccccaggaataaaagcattttgctacatcaattgggattgggaatattggtctaacagaaatggttttccatggcatgattggaaagatgcacgtatcgaaaaaaatccatttgtattagaggcttataaaaaagaaatggagaagccattatttattcaccttaataataaataa